(19) United States
(12) Patent Application Publication
(10) Pub. No.: US 2026/0233003 A1
(43) Pub. Date: Aug. 13, 2026

(54) ELECTRONIC DEVICE AND METHOD

(71) Applicant: Sony Group Corporation, Tokyo (JP)

(72) Inventor: Diederik Paul MOEYS, Stuttgart (DE)

(73) Assignee: Sony Group Corporation, Tokyo (JP)

(21) Appl. No.: 19/154,404

(22) PCT Filed: Feb. 16, 2024
(86) PCT No.: PCT/EP2024/054040

§ 371 Date: Aug. 7, 2025

(30) Foreign Application Priority Data
Feb. 24, 2023 (EP) 23158553.0

(51) Int. Cl. A61N 1/36; A61N 2/00; A61N 7/00

(52) U.S. Cl. CPC A61N 1/36036; A61N 1/36031; A61N 2/004; A61N 7/00

(57) ABSTRACT

An electronic device comprising circuitry configured to generate a vestibular stimulus based on motion sequences included in control information and to perform vestibular stimulation based on the vestibular stimulus.

### TECHNICAL FIELD

The present disclosure pertains to an electronic device configured to stimulate a user's vestibular organ to perform motion rehearsal.

### TECHNICAL BACKGROUND

Senses generally accepted to be accessible to human perception include the vestibular sense, generally known as the sense of balance. This sense is associated with the vestibular system, located within the human cranium in the region of the inner ear, adjacent to the cochlea on each side. Specifically, the vestibular system consists of two sensory arrangements generally assumed to be responsible for evoking a sense of rotation and a sense of linear acceleration respectively. A sense of rotation is generated by an arrangement of three semicircular canals approximately arranged in three linearly independent spacial planes (on each side of the cranium). These canals are filled with a fluid that, if the cranium, and thus the canals, are rotated about an axis, interacts with a set of hair cells present on the inside of the canals and which, if interacted with, evoke a sense of rotation in a person. A sense of linear acceleration is generated by a set of otoliths also included in the vestibular system. Artificial stimulation of these structures, the semicircular canals and the otoliths, or evocation of an associated nerve response can be accomplished through various means, with initial experiments utilizing transcranial direct current stimulation (tDCS) dating back to the 18^{th} century.

During athletic training, the athlete is often required to perform complex or difficult motions in order to achieve the desired result. These motions need to be rehearsed. These rehearsals are intended not only for the athlete to get accustomed to the movement of their limbs and muscles in the motion, but also to the feeling of the motion being performed. The feeling of the motion being performed includes stimulation of the vestibular system. Having a vestibular sense accustomed to the vestibular stimulation felt during the motion can improve the athlete's ability to execute the motion in subsequent attempts. However, as natural stimulation of the vestibular system generally requires performing the motion, which carries with it inherent complexity and, depending on the nature of the movement, danger. There is therefore a need for a method or device to provide an artificial stimulation akin to that felt during the performance of a motion without actually performing the motion.

There are devices that include stimulation of the vestibular system to provide, for example, stimuli to accompany visual stimuli, as described in patent document 1. Moreover, there are devices that stimulate a patient's vestibular system in order to augment or control a patient's respiratory function, open the patient's airway, induce sleep, and/or counteract vertigo, such as described in patent document 2. There are furthermore systems and methods for game playing using vestibular stimulation that include detecting motions associated with the user by a feedback sensor device and providing motion information from the feedback sensor device to a game device, such as described in patent document 3.

Vestibular stimulation has also been shown to allow stimulation along multiple special axes, specifically in a virtual reality setting, such as described in the research paper “Omnidirectional Galvanic Vestibular Stimulation in Virtual Reality” by Groth et al., published IEEE: Transactions on Visualization and Computer Graphics 2022.

### LIST OF REFERENCES

Patent document 1: United States Patent Nr. 11458313 B1
Patent document 2: United States Patent Application Nr. 20080275513 A1
Patent document 3: United States Patent Application Nr. 20100113150 A1

### SUMMARY

According to a first aspect, as set forth in independent claim 1, the disclosure provides an electronic device comprising circuitry configured to generate a vestibular stimulus based on motion sequences included in control information and to perform vestibular stimulation based on the vestibular stimulus.

According to a second aspect, as set forth in independent claim 23, the disclosure provides method to generate a vestibular stimulus based on motion sequences included in control information and to perform vestibular stimulation based on the vestibular stimulus.

Further aspects are set forth in the dependent claims, the drawings and the following description.

### BRIEF DESCRIPTION OF THE DRAWINGS

Embodiments are explained by way of example with respect to the accompanying drawings, in which:

FIG. 1 is an illustration of a lateral view of a user's head showing the approximate positioning of one embodiment of a device according to the present disclosure in relation to the user's vestibular system; and

FIG. 2 is an illustration of the semicircular canals of the vestibular system and an illustration of a cross section of the semicircular canals; and

FIG. 3a is an illustration of a lateral view of a user's head showing the approximate positioning of one embodiment of a device according to the present disclosure in relation to the user's head; and

FIG. 3b is an illustration of a frontal view of a user's head showing the approximate positioning of one embodiment of a device according to the present disclosure in relation to the user's head; and

FIG. 4a is an illustration of one embodiment of a device according to the present disclosure and a stimulus based on ultrasound stimulating a user's vestibular system; and

FIG. 4b is an illustration of one embodiment of a device according to the present disclosure and a stimulus based on (directed) electromagnetic waves stimulating a user's vestibular system; and

FIG. 5 is an illustration of the principal algorithm of one embodiment; and

FIG. 6a is an illustration of the application of a stimulus in one direction according to one embodiment; and

FIG. 6b is an illustration of the application of a stimulus in a second direction according to one embodiment; and

FIG. 7a is an illustration of the computation of the stimulus based on an intended direction according to one embodiment; and

FIG. 7b is an illustration of another computation of the stimulus based on an intended direction according to one embodiment; and

FIG. 8 is an illustration of motion sequences being included in the control information according to one embodiment; and

FIG. 9 is an illustration of an algorithm to determine a timed sequence of stimuli based on the motion sequences according to one embodiment; and

FIG. 10 is an illustration of an algorithm to determine a timed sequence of the strength of stimuli based on the motion sequences according to one embodiment; and

FIG. 11 is an illustration of an algorithm to generate a sequence of stimuli with concurrent video output based on a selected motion sequence according to one embodiment; and

FIG. 12 is an illustration of an algorithm to acquire feedback according to one embodiment; and

FIG. 13 is an illustration of an algorithm to acquire a motion sequence based on an athletic performance; and

FIG. 14 is an illustration of an algorithm to acquire a calibration according to one embodiment; and

FIG. 15 is an illustration of an algorithm to acquire a calibration according to one embodiment; and

FIG. 16 is an illustration of an algorithm to acquire a calibration according to one embodiment; and

FIG. 17 is an illustration of an algorithm to acquire a calibration using machine learning according to one embodiment; and

FIG. 18 is an illustration of an algorithm to further control the stimulus based on eye movement; and

FIG. 19 is an illustration of a circuit according to the present disclosure in one embodiment.

### DETAILED DESCRIPTION OF EMBODIMENTS

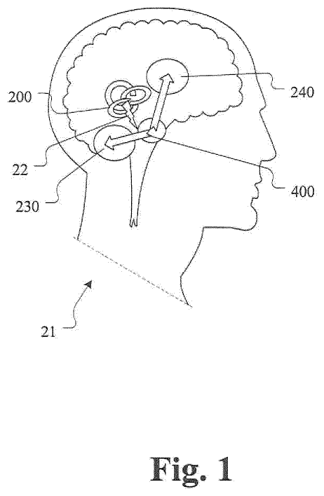

Before a detailed description of the embodiments under reference of FIG. 1 is given, general explanations are made.

Some embodiments of the present disclosure provide an electronic device comprising circuitry configured to generate a vestibular stimulus based on motion sequences included in control information and to perform vestibular stimulation based on the vestibular stimulus.

Circuitry may include a processor, a memory (RAM, ROM or the like), a storage, input means (keyboard, camera, etc.), output means (display (e.g. liquid crystal, (organic) light emitting diode, etc.), loudspeakers, etc., a (wireless) interface, etc., as it is generally known for electronic devices (computers, smartphones, etc.). Moreover, it may include sensors for sensing still image or video image data (image sensor, camera sensor, video sensor, etc.), for sensing a fingerprint, for sensing environmental parameters (e.g. radar, humidity, light, temperature), etc.

A stimulus can be understood to be generated by the circuitry and can be a signal directed towards the vestibular system of the user. The stimulus can be provided by a stimulator or stimulation assembly integrated in the circuitry. The stimulus can be a stimulation or stimulation signal generated by the circuitry or the stimulator and can be continuous or discontinuous, it can also be modulated by an appropriate method, including Pulse Width Modulation. “Stimuli” denotes the plural of the word “stimulus”. A stimulation is stimulation is the stimulation of the vestibular system such that a feeling or sense of the vestibular system being stimulated is evoked in the user. The control information can include one or more motion sequences that can be used to generate vestibular stimuli. “Motion sequences” denote a plurality of “motion sequence”. The motion sequence is used as a basis for generating the vestibular stimuli. The motion sequence describes the vestibular stimuli felt during a motion.

Some embodiments of the present disclosure further provide that the control information includes timing information. The timing information can be included in the motion sequences, such that the vestibular stimuli can be generated based on the motion sequences starting at a time between the start and the end of the motion sequence as requested by, for example, user input.

The timing information can also be used to adjust a rate at which the vestibular stimuli are being generated. The timing information can also be associated with the entirety of the motion sequences, such that certain motion sequences are used for generating the vestibular stimuli at certain times.

Apart from timing information, the motion sequences can include location information indicating the location of the user, or position or pose information, indicating the position or pose that the user is assuming, such that a motion sequence appropriate for the position or pose is chosen.

Some embodiments of the present disclosure further provide that the circuitry is configured to hold the control information and the motion sequences include information on timed sequences of intended stimuli that controls the generation of the vestibular stimulus. A motion sequence can therefore include a sequence of stimuli the user is intended to be stimulated with in sequence. The motion sequence can be based on a motion of a person or an athlete performing a sports motion. Control information can be data stored on an electronic storage device included in the circuitry or can be received from a remote device.

Some embodiments of the present disclosure further provide that motion sequences can be selected by a user input or based on user preference information. The user can provide input to the device in order to choose the motion sequence on the basis of which the vestibular stimulus should be generated. For example, the user may wish to sense the motion felt while swinging a golf club, a rotation during high diving or throwing a bowling ball or the vestibular stimulation felt during an aerobatics routine. An appropriate motion sequence can be provided and the user can, by user input, choose the motion sequence. Another embodiment envisions the motion sequences to be chosen based on user preference information. The user may, for example, provide user preference information indicating that vestibular stimulations of golf swings in general should be provided. Then the device can choose different motion sequences associated with different golf swings to be used in generation of the vestibular stimuli. The provided example is only illustrative and non-limiting. Other ways of using user preference information for selection of motion sequences will be readily apparent to the skilled person.

Some embodiments of the present disclosure further provide that is further configured for the user to perform motion rehearsal and/or accustomization based on vestibular stimulation. The user can use the device become accustomed to the vestibular stimulation felt during a motion while sitting or performing preparatory exercises. The user can also become accustomed or the vestibular stimulation while assuming an initial position or pose of the sports motion or a position or pose assumed while performing the motion without actually performing the motion. As certain motions or sports motions may be inherently difficult or highly technical to execute or dangerous, this method can be used to rehearse the feeling felt when actually performing the motion is safety. This can also prevent unwanted reactions of the unaccustomed vestibular sense while actually performing the motion, which can increase safety or the accuracy of t In a third step S531 the user 21 is requested to provide input to determine whether the vestibular stimulus 22 is felt. he performance. The method can also increase immersion, if the user wishes to rehearse the sports motion, for example, in addition to VR (virtual reality) or XR (extended reality). The method can also reduce virtually induced motion sickness (VIMS), which is caused by a discrepancy between the information conveyed by the visual sense and the vestibular sense in the same setting. The user can also visualize the motion with closed eyes, for example. The method can also be used for rehabilitation with the user getting reaccustomed to certain movements after periods of inactivity. The rehabilitation may, for an athlete, be required during a training break following an injury. The rehabilitation may also, for example, be required for a person during reconvalescense in a lying or sitting position during sickness or following an accident.

Some embodiments of the present disclosure further provide that the motion sequences describe a difficult and/or technical motion and the motion is a sports motion. The motion can be a sports motion. Each motion sequence can be used to generate vestibular stimuli individually. The vestibular stimuli can be generated based on the motion sequences slower of faster than the motion they are based on, such that the user feels the motion to take longer or shorter than while actually being performed. Using the device, the vestibular stimulation felt by a person or athlete performing a motion, such as a sports motion, can be evoked in the user. This device therefore makes it possible to feel the vestibular stimulation that a person or athlete would feel while performing the motion without actually performing the motion. Instead, the vestibular stimulus can be felt while sitting, standing or assuming a static position that the body would assume at one time while performing the motion.

Some embodiments of the present disclosure further provide that the motion sequences are obtained by inertial measurement of the movement and/or the acceleration of the motion of an athlete. In order to obtain the motion sequences, the acceleration and movement of the athlete performing the motion is recorded, as described hereinbelow according to one embodiment. The movement and acceleration of the athlete can be recorded by an external device including appropriate sensors, such as an accelerometer or an inertial sensor, but may also be based on imaging as captured by an appropriate imaging device. The motion sequence can then, for example, include intended stimuli derived from the motion. If, for example, the athlete is rotating in a left direction, then the motion sequence can include a left rotation as the intended stimulus. The motion can also only be a motion of the head of the athlete. There can be embodiments where the device according to the present disclosure is used to record the movement of the athlete. This way, the user, who may be the athlete, can generate the motion sequence by recording his own movement and acceleration with the device.

Some embodiments of the present disclosure further provide that the vestibular stimulation is a calibrated vestibular stimulation. A calibrated stimulation is a stimulation that is evoked by a stimulus that is modified by a calibration. A calibration can be understood to mean that a calibration measurement is taken that will cause the vestibular stimuli to generated according to sensitivity of the user to vestibular stimulation. Provision of a calibrated stimulation can enhance the precision of the stimulus to achieve the intended stimulation of the user's vestibular system. This is to ensure that the stimulus is of the required strength and direction for the user to sense. This is also to ensure that the generated stimulus is not of a strength that will overwhelm the user's vestibular system or cause discomfort.

Some embodiments of the present disclosure further provide that the circuitry is configured to further compute the calibrated vestibular stimulus based on a calibration obtained from the user. The device can be configured to allow the user to enter a calibration mode in order to obtain the calibration. A vestibular stimulus can then be generated and the user asked to provide an input indicating whether, and in what strength, the stimulus is felt. The stimulus and the input can then be used to generate the calibration. There are also embodiments where the calibration is obtained by sensing a user reaction. This can be accomplished by generating a stimulus and then sense a change of pose, position or posture of the user.

Some embodiments of the present disclosure further provide that the circuitry is configured to obtain the calibration using machine learning. This may entail the device to enter a calibration mode or may be accomplished during normal operations. Machine learning may also be used in sequence or in parallel to calibration based on user input.

Some embodiments of the present disclosure further provide that the circuitry is configured to generate the vestibular stimulus at various levels of intensity. The intensity can be the strength of the stimulus as generated based on information on the intended strength of the stimulus as included in the motion sequences. The intensity can be modified based on the calibration.

Some embodiments of the present disclosure further provide that the circuitry is configured to further track position and/or posture of a user with at least one camera and/or EEG, and/or body trackers to estimate the pose of the user. The EEG is an electroencephalograph that may be either included in the circuitry or provide information to the circuitry remotely. The pose may be the position of the user in space or a posture of the body. Body tracking means, such as a body tracking device, may provide information to the circuitry remotely or be included in the circuitry. The camera may be included in the circuitry monitoring the body of the user or the surrounding of the user. The camera may also be included in an external device that provides image or video information to the circuitry remotely, monitoring the body of the user. Some embodiments may, in addition to a camera, EEG or body tracking, use other sensing means to acquire further biosignatures such as a temperature, a skin conductance and other. Some embodiments of the present disclosure use the information thus obtained to generate the calibration or to generate the motion sequences. There are also embodiments that use the estimation of the pose of the user as a basis for generating feedback for the circuitry, why may be used to modify the generation of the vestibular stimuli in subsequent stimulation based on the motion sequences. Yet other embodiments are envisaged wherein the estimation of the pose of the user is used to provide feedback to the user. For example, if the user wishes to assume a pose or position of a sports motion, the device could provide feedback on whether the pose or position is assumed and held correctly. The feedback in this case could, for example, include visual, acoustic or vibrational signals. The cameras may, for example, be an event based camera or Event-based Vision Sensor (EVS).

Some embodiments of the present disclosure further provide that the circuitry is configured to control the generation of the vestibular stimulus based on an eye movement. The eye movement can be the relative movement of the pupil in the eye of the user, but can also be an eye gaze. Eye movement can be tracked by an eye tracking device included in the device. By tracking eye movement it can be determined if the user is losing concentration which rehearsing a movement, is feeling discomfort or may be intending to interrupt the stimulation. For example, eye movement towards the edge of the field of vision of the user may indicate loss of concentration.

Some embodiments of the present disclosure provide that the circuitry is further configured to interrupt the stimulation based on the eye movement. Some embodiments provide for the stimulation to be interrupted if eye movement towards the edge of the field of vision of the user is detected. Other embodiments may provide for the strength of the stimulus to be increased or decreased or the direction of the stimulus to be altered.

Some embodiments of the present disclosure provide that the motion sequences are obtained during a training process in advance. Motion sequences can be set in advance and act in the manner of a recording of vestibular stimuli to be reproduced like audio tracks act like a recording of sounds to be reproduced. Some embodiments provide for the motion sequences to be provided to the circuitry during manufacture.

Some embodiments of the present disclosure provide that the motion sequences are obtained by machine learning. Obtaining the motion sequences as described hereinabove may include use of a machine learning algorithm or deep neural network.

Some embodiments of the present disclosure provide that the circuitry is further configured to cause a video display apparatus to display, to the user, video information associated with the motion sequences. For example the video information may visualize the same motion as the motion sequence that describes the vestibular stimulus. The video display apparatus may, for example, be a virtual reality headset, an extended reality device or an augmented reality device. The video display apparatus may also be a television screen, a computer monitor or the display of a cell phone or a smart phone or any other device capable of displaying images electronically. The user can, for example, rehearse the motion while, based on the motion sequence, video information is displayed. The user can, for example, rehearse the vestibular stimulation felt during an aerobatics routine while observing the point of view of a pilot executing the aerobatics routine. The video can further be blurred in order to indicate acceleration, which can increase the perceived (though not the) strength of the vestibular stimulus and increase the effectiveness of the stimulation based on the motion sequence.

Some embodiments of the present disclosure provide that the circuitry is further configured to stimulate, with the vestibular stimulus, at least one semicircular canal of the user's vestibular system or at least one otolithic organ of the user's vestibular system or at least one semicircular canal and at least one otolithic organ of the user's vestibular system. It should be noted that stimulation of the semicircular canals will cause the user to feel a rotational motion whereas stimulation of the otolithic system will cause the user to feel a linear acceleration. Either system can be stimulated using the methods described herein.

Some embodiments of the present disclosure provide that the circuitry is further configured to stimulate the user's vestibular system by at least one of a direct current stimulation, voltage stimulation, electromagnetic or magnetic stimulation, ultrasonic stimulation or direct nervous or vestibular stimulation. The stimulator, generating the stimulus that stimulates the vestibular system of the user, may be an assembly consisting of a cathode and an anode provided in one or more separate housings such that a direct current can be applied to the user's cranium. The direct current may be applied at a precise voltage. The stimulator may also be device capable of generating an ultrasonic wave signal that may be directed or isotropic. The stimulator may also be an electromagnetic or magnetic wave emitter that emits directed or isotropic waves. Electromagnetic or magnetic stimulation can be accomplished with beam-steering or coils. The stimulator may also be a stimulator that is connected directly to the user's vestibular nerves. The stimulator may be provided such that the stimulus is provided in the inner ear. The stimulator may further, for example, act as an interface of the electronic device to the user's vestibular system.

Some embodiments of the present disclosure, as described hereinabove, provide that the circuitry comprises an EEG device or body-tracking means configured to sense the user reaction. The EEG, camera or body tracking means may be included in the circuitry.

Some embodiments of the present disclosure provide that the circuitry is provided in one or more head-mounted casings to be worn by the user. As the vestibular system is located in the head, providing the device in one or more head-mounted casings is useful. However, since some modes of vestibular stimulation, such as stimulation with magnetic or electromagnetic waves, may be provided remotely, some embodiments of the present disclosure may deviate from the head-mounted arrangement.

Some embodiments of the present disclosure provide that the circuitry is provided in one or more individual casings, or is incorporated into headphones, or is incorporated into a virtual-reality headset, or is incorporated into an extended-reality device. The device may also be incorporated in other types of headwear, such as hats, helmets, in-ear headphones and others.

Some embodiments of the present disclosure provide for a method comprising generation of a vestibular stimulus based on motion sequences included in the control information and to perform the vestibular stimulation based on a vestibular stimulus.

The methods as described herein are also implemented in some embodiments as a computer program causing a computer and/or a processor to perform the method, when being carried out on the computer and/or processor. In some embodiments, also a non-transitory computer-readable recording medium is provided that stores therein a computer program product, which, when executed by a processor, such as the processor described above, causes the methods described herein to be performed.

Returning to FIG. 1, as illustrated, the vestibular system 200 of a user 21 is being stimulated by an (artificial) vestibular stimulus 22, leading to a consciously perceptible sensation 240 and a unconscious reaction 230. The consciously perceptible sensation 240 evokes a feeling of either being linearly accelerated in a given direction or rotating about a given axis, even while the user 21, or -more precisely-the user's vestibular system 200 remains motionless. The vestibular system 200 is located inside the human skull in the area of the ears on either side.

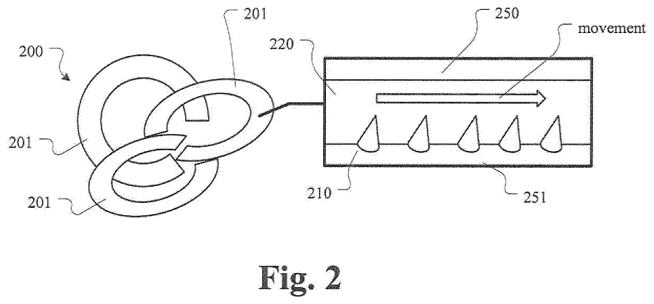

FIG. 2 shows as a mode of natural stimulation of the vestibular system. The vestibular system 200 is located in two distinct areas on both sides of the human skull. It includes three so-called semicircular canals 201, arranged in three linearly independent special planes. Each semicircular canal 201 is formed by channel-like tubes 250 in a ring shape. Each semicircular canal contains a fluid 220. If the vestibular system 200 (and, by extension, the user's head) is rotated about an axis, by inertia, the fluid 220 is displaced with respect to the tube 250. Set in the wall 251 of the tube 250 are a series of hairs 210, that are deflected by the displaced fluid 220. This deflection leads to a nerve signal that is interpreted by the nervous system of the user 21 as (in the case of the semicircular canals) rotation about an axis. Linear acceleration is sensed by stimulation of the otolithic organs (not shown).

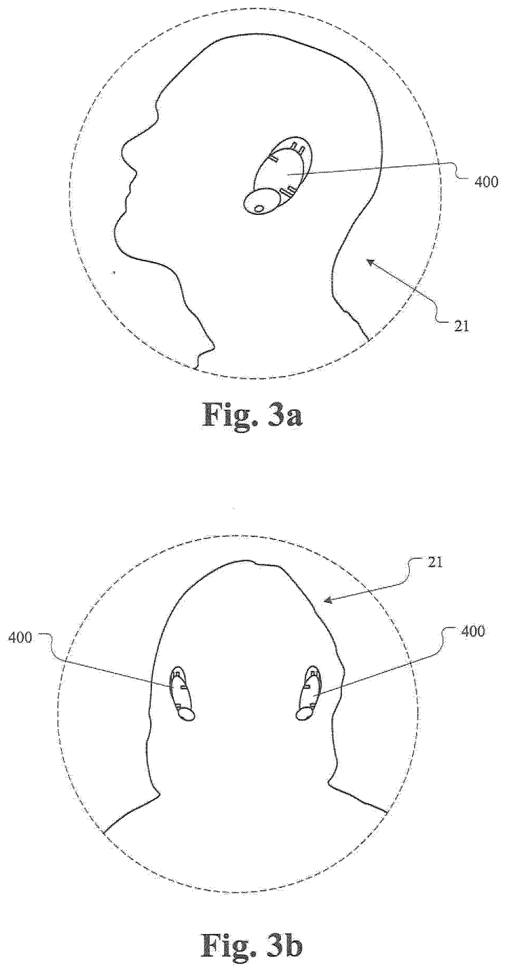

In FIG. 3a and FIG. 3b, an arrangement of devices according to one embodiment is shown in a lateral and a frontal view of the user 21. Stimulation of the vestibular system, can be accomplished by a device set in a series of stimulators 400 worn on the user's head close to the ears, as shown in FIG. 3a, on either side, as shown in FIG. 3b. Arrangements exist where the device is instead provided in different locations on a user's head, such as the neck, the forehead or the crown. Other embodiments exist where the number of positions and/or stimulators 400 is larger, such as four or six. Different arrangements, positions and larger number of positions can allow for stimulations of different semicircular canals 201, leading to different sensations.

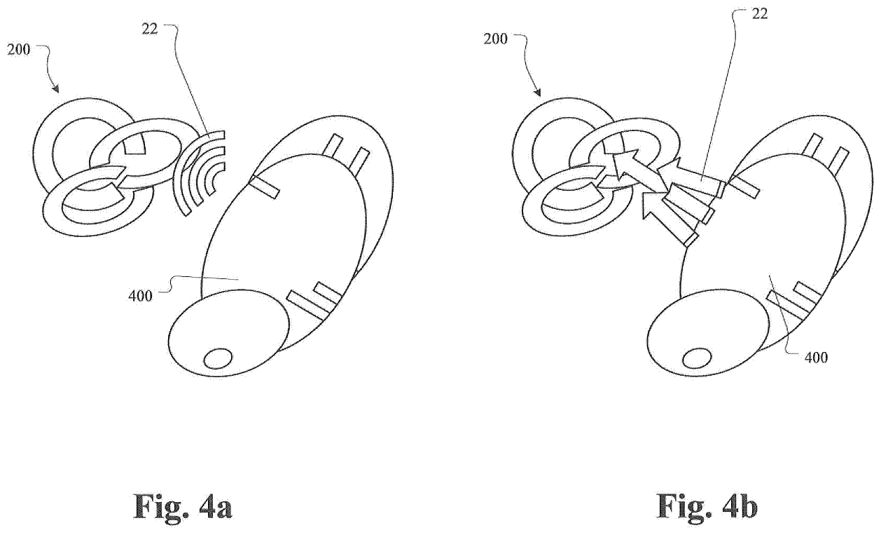

In FIG. 4a and FIG. 4b, different types of stimuli 22 are illustrated. FIG. 41 shows stimulation of a vestibular system 200 by an ultrasonic vestibular stimulus 22 generated by the stimulator 400.

FIG. 4b shows stimulation of a vestibular system 200 by an electromagnetic wave vestibular stimulus 22 or a magnetic vestibular stimulus 22 generated by the stimulator 400. Note that, in FIG. 4b, the electromagnetic vestibular stimulus 22 may be directed towards the vestibular system 200 through beam-steering.

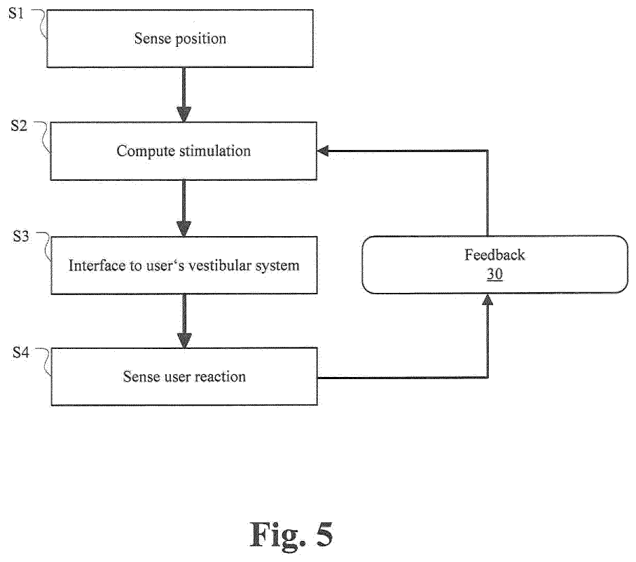

FIG. 5 shows one iteration of main algorithm according to one embodiment. In an optional first step S1 a pose 25 of the user 21 is sensed through the use of an appropriate position sensing method or device, such as described hereinabove. In a second step S2, based on the pose 25, the appropriate vestibular stimulus 22 is computed. The vestibular stimulus 22 is then generated by the stimulator 400 and emitted in a third step S3. In an optional fourth step S4, a user reaction is sensed. The user reaction may, for example, include a change in pose 25. The user reaction is then, in some embodiments, processed (also in S4), and optional feedback 30 is generated. The feedback 30 can, for example, be an indicator to the user based on the user reaction. The indicator can, for example, be an acoustic signal or a vibrational signal. The algorithm is iterated until, for example, the end of a motion sequence 29 is reached or the stimulation task is interrupted by user input or eye movement.

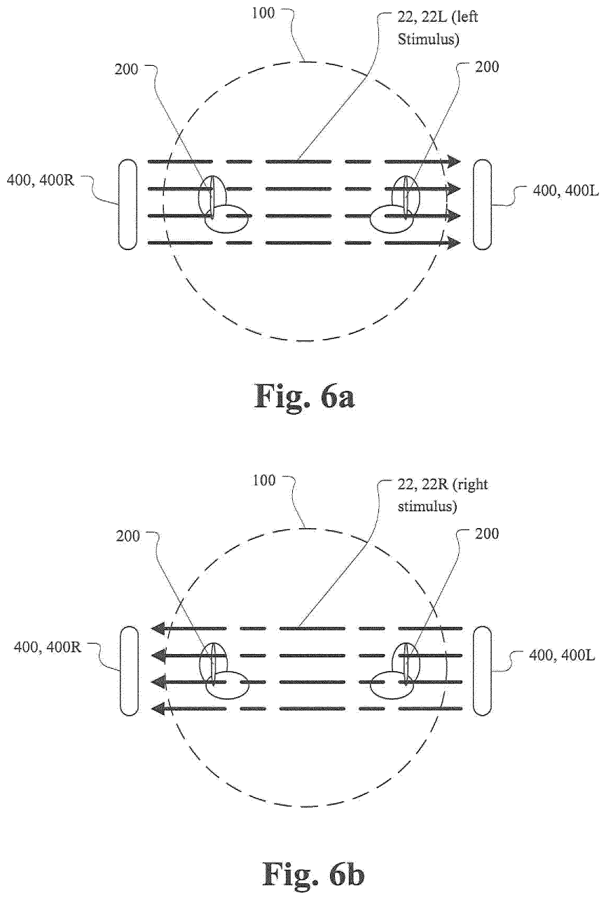

One method to generate a vestibular stimulus 22 as a left vestibular stimulus 22L or a right vestibular stimulus 22R according to one embodiment is illustrated in FIG. 6a and FIG. 6b. Returning to FIG. 6a, the vestibular stimulus 22L is generated for example by a stimulator 400R on the right side and another stimulator 400L on the left side arranged on either side of a cranium, 100 such that a vestibular stimulus 22 can be transmitted from the stimulator 400R to the stimulator 400L while passing through the vestibular systems 200. The vestibular systems 200 can be the vestibular systems 200 of the user 21. The vestibular stimulus 22L can, for example be a direct electrical current running from the stimulator 400R as a cathode to the stimulator 400L as an anode. This causes the vestibular systems 200 to be stimulated such that the user 21 senses a rotation or linear acceleration to the left.

Continuing to FIG. 6b, a stimulation to the right side 22R can be generated by reversing the electrical current with the stimulator 400R acting as an anode and the stimulator 400L acting as an anode. This causes the vestibular systems 200 to be stimulated such that the user 21 senses a rotation or linear acceleration to the right.

However, the stimulation 22 does not have to rely on direct current stimulation as described. Instead, the stimulation 22 may instead be accomplished by generation of an ultrasonic wave by the stimulators 400. The stimulation can also be accomplished by generation of an electromagnetic or magnetic wave or beam. The wave or beam can be directed via beam-steering, prearranged interference-patterns or appropriate antenna arrangements. If accomplished via generation of a direct current, the stimulators may be attached on the outside of the cranium 100 of the user, but may also be provided in an ear canal of the user 21.

The stimulation can also be generated by direct stimulation of the vestibular nerve of the user 21. A stimulator of this type may be surgically implanted into, or into the vicinity of, the vestibular system 200 of the user 21. This arrangement can be useful if the user's 21 vestibular system, particularly the semicircular canals 201 or the otolithic system is damaged.

In any case, in order to maintain a save level of stimulation, the strength of the vestibular stimulus 22 should be limited. The limit for direct current stimulation can, for example, be 10 mA. The strength of the vestibular stimulus can, in some embodiments, be determined by the device, as described further hereinbelow. The vestibular stimulus can, as described hereinabove, be continuous or discontinuous, it can also be modulated by an appropriate method, including Pulse Width Modulation.

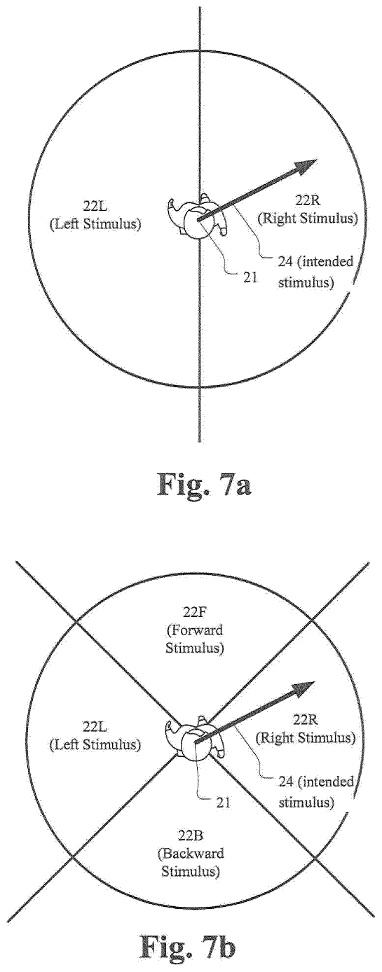

FIGS. 7a and 7b show the determination of the vestibular stimulus 22 in a local coordinate system of the user 21. The user 21 is facing forward. Then, in the embodiment shown in FIG. 7a, about an axis extending from the surface the user is located on, upwards, the surrounding of the user 21 can be divided into a left sector and a right sector. If an intended stimulus 24 points in a direction located in the right sector (as shown), then a vestibular stimulus in the right direction 22R can be generated. If, on the other hand, the intended stimulus 24 points in a direction located in the left sector, then a vestibular stimulus in the left direction 22L can be generated. There can be embodiments where a vestibular stimulus 22 is generated if the intended stimulus 24 points in a direction in the right sector, but no vestibular stimulus 22 is generated if the intended stimulus 24 points in a direction in the left sector, or vice versa. There can also be embodiments wherein generation of a vestibular stimulus 22 further depends on parameters, such as the pose 25, feedback 30, a surrounding of the user 21 as determined by a timing, imagery, video or others.

There can also be embodiments where a vestibular stimulus in a left direction 22L can be generated despite the intended stimulus 24 pointing in a direction in the right sector or vice versa. The determination of which vestibular stimulus 22 is generated can likewise be based on parameters, such as the pose 25, feedback 30, a surrounding of the user 21 as determined by imagery, video or others.

FIG. 7b shows an embodiment wherein the surrounding of the user is divided into four distinct sectors, namely a left, right, forward and backward sector. Here, as in the embodiment shown in FIG. 7a, if the intended stimulus 24 points in a direction in the right sector, a vestibular stimulus in a right direction 22R can be generated or, if the intended stimulus 24 points in a direction in the left sector, a vestibular stimulus in a left direction 22L can be generated. If the intended stimulus 24 points in a direction in the forward sector, a vestibular stimulus in a forward direction 22F can be generated or, if the intended stimulus 24 points in a direction in the backward sector, a vestibular stimulus in a backward direction 22B can be generated. There are embodiments wherein, with the intended stimulus 24 pointing in a direction in a particular sector, no vestibular stimulus 22 is generated. For example, a vestibular stimulus 22 can be generated if the intended stimulus 24 points in a direction in the backward sector, but no vestibular stimulus is generated if the intended stimulus 24 points in a direction in the forward sector. This is possible in all combination of sectors. As described with respect to FIG. 7a, there can also be embodiments where a vestibular stimulus in a one direction can be generated despite the intended stimulus 24 pointing in a direction in a different sector. Again, the determination of which vestibular stimulus 22 is generated can likewise be based on parameters, such as the pose 25, feedback 30, a surrounding of the user 21 as determined by a timing, imagery, video or others.

It should be noted that the number or arrangement of the sectors can be different from the embodiments illustrated in FIG. 7a and FIG. 7b. For example, there can be an odd number of sectors or a larger number of sectors. The size of the sectors can also be different between each other. There can be embodiments with sectors arranged around a different axis or sectors arranged outside of a plane. Sectors can subtend a solid angle as opposed to a plane angle. The number, shape, size and arrangement of the sectors can be determined based on parameters, such as the pose 25, feedback 30, a surrounding of the user 21 as determined by a timing, imagery, video or others.

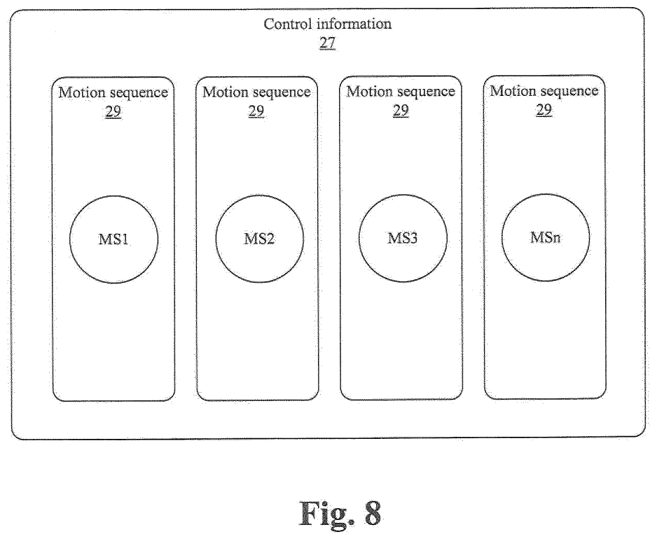

FIG. 8 illustrates control information 27 including one or more motion sequences 29 according to one embodiment. Each motion sequence 29 can contain timed sequences of intended stimuli 24. Timed sequences of intended stimuli 24 contained in one motion sequence 29, for example motion sequence 29,MS1, can be executed individually. Which motion sequence 29 is executed may be determined by user input. For example, the user 21 can be provided with a display with an arrangement similar to the illustration in FIG. 8, wherein a number of motion sequences 29 are displayed as selectable options and the user 21 can individually choose one of the selectable options, which then causes, according to the timed sequences of intended stimuli 24 included therein, sequences of vestibular stimuli 22 to be generated by the stimulator 400.

Each motion sequence 29 can contain intended stimuli 24 describing the vestibular stimulation felt during a sports motion. A sports motion can, for example, be a short motion such as swinging a golf club, a rotation during high diving or throwing a bowling ball. The motions can also be longer motions such as an aerobatics routine, a car race or alpine skiing. The choice of which motion sequence 29 is selected can also be aided or taken based on user preference information. Individual motion sequences 29 may be stored on storage medium provided in the circuitry, but may also be accessed remotely.

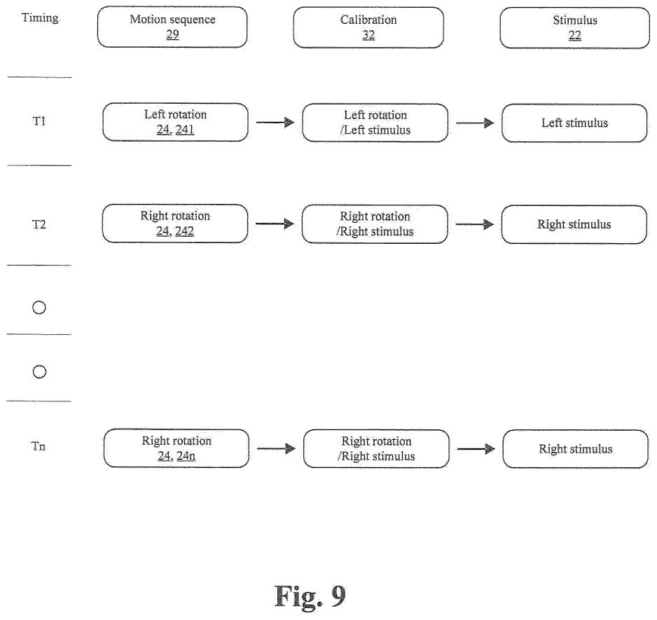

FIG. 9 illustrates another embodiment of the determination of a stimulus 22 based on a motion sequence 29 contained in the control information 27, including a calibration 32. The motion sequence 29 may be selected as described hereinabove. Here, as described hereinabove, the motion sequence 29 contains sequences of intended stimuli 24 associated with timing information. For example at the time T1 can be a time elapsed since the start of the motion sequence 29. However, the timing T1 can in addition indicate the temporal coordinate of a time window. The time window can, for example, have a duration of 1 ms (milli-second), though other lengths, such as 0.1 s (second) are possible. The timing T1 is associated with an intended stimulus 24, 241, which, for example, can indicate “left rotation”. This would indicate that, at the time T1, as determined, for example, by a system clock, the intended stimulus 24 is “left rotation” for a duration of 1 ms. After the time window associated with T1 has elapsed, a second time window associated with a timing T2 is entered. This would indicate that, for a second duration of 1 ms, the intended stimulus 24,242 is “right rotation”. The intended stimulus 24 thus obtained is then compared with the calibration 32. The calibration 32 can, for example, contain associated intended stimuli 24 and vestibular stimuli 22. For example, at the timing T1, the “left rotation” obtained from the intended stimulus 24 is associated with a left stimulus. The left stimulus can be the left stimulus 22L as illustrated in FIG. 6a, FIG. 6b, FIG. 7a and FIG. 7b. Analogously, a right stimulus can be the right stimulus 22R as illustrated in FIG. 6a, FIG. 6b, FIG. 7a and FIG. 7b and a forward stimulus can be the forward stimulus 22L etc.

Furthermore, forward rotation and backward rotation are possible, as are superposed rotations, such as a combined left-and-forward rotation or a right-and-backward rotation and all other combination of rotational directions. The rotational direction can also be given in terms of a vector in space, measured in the coordinate system of the user or in the coordinate system of the space the user is in and can have any orientation. The intended stimulus 24 can also describe linear acceleration along any axis. The sequence of intended stimuli included in the control information 27 is executed until the sequence terminates at a timing Tn, where n is the number of timings contained in the control information 27. The vestibular stimulus 22 obtained via the calibration 32 can be called a calibrated vestibular stimulus. However, the inclusion of a calibration 32 is optional. Instead, the vestibular stimulus 22 may be generated based on the intended stimulus 24 without involvement of the calibration 32.

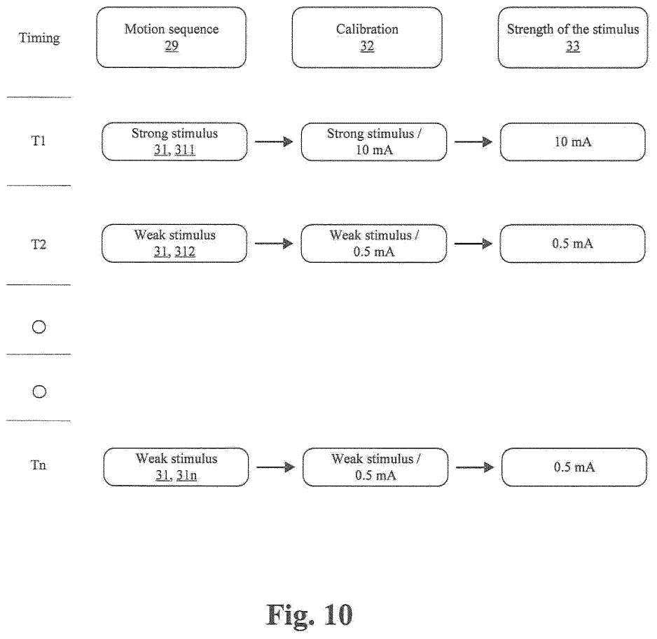

FIG. 10 illustrates another embodiment of the determination of a strength of a stimulus 33 based on a motion sequence 29 contained in the control information 27, including a calibration 32. The motion sequence 29 may be selected as described hereinabove. Here, in addition to sequences of intended stimuli 24, the control information 27 includes sequences of intended strengths of stimuli 31, equally associated with the timing information. The timing T1 is further associated with an intended strength of the stimulus 31, 311, which, for example, can indicate “strong stimulus”. This would indicate that, at the time T1, as determined, for example, by a system clock, the intended strength of the stimulus 31 is “strong stimulus” for a duration of 1 ms. We reiterate that the intended strength of the stimulus 31 can also be associated with an intended stimulus 24. Thus, the “strong stimulus” as illustrated, may be associated with “right rotation” at T1, such that a “strong stimulus” and a “right rotation” are obtained. After the time window associated with T1 has elapsed, the second time window associated with a timing T2 is entered. This would indicate that, for a second duration of 1 ms, the intended strength of the stimulus 31,312 is “weak stimulus”. The intended strength of the stimulus 31 thus obtained is then compared with the calibration 32. The calibration 32 can, for example, contain associated intended strength of the stimuli 31 and strengths of the stimuli 33. The strength of the stimulus 33 can, for example, be a voltage, if the stimulator is a direct current stimulator. For example, “strong stimulus” can, for example, be associated with 10 mA, causing the stimulus to be generated with a strength of 10 mA. Likewise, a “weak stimulus” can, for example, be associated with a voltage of 0.5 mA.

The sequence of intended stimuli included in the motion sequence 29 is executed until the sequence terminates at a timing Tn, where n is the number of timings contained in the control information 27.

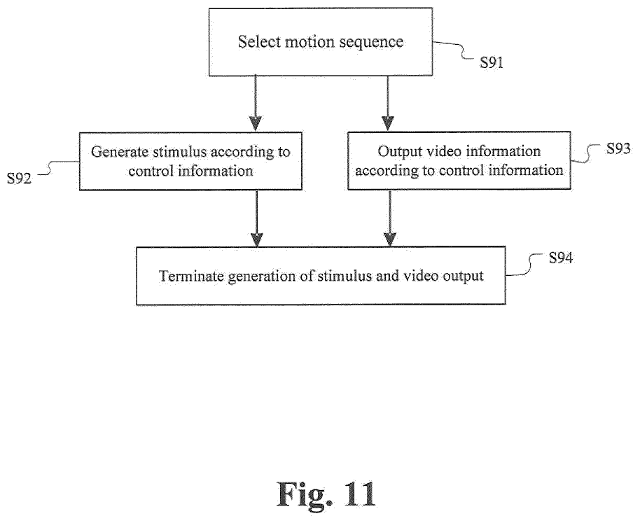

FIG. 11 illustrates one aspect of some embodiments of the present disclosure, namely output of video information associated with the motion sequences 29. For example, in a first step S91, a motion sequence 29 is selected. In a second step S92, a stimulus 22 as described hereinabove for example according to the embodiments illustrated in FIG. 9 and FIG. 10, is generated.

Concurrently, in a third step S93, video information associated with the motion sequence 29 is displayed to the user 21 by appropriate image generation means. Thus, for example, a video showing the point of view of an athlete performing a high dive can be displayed to the user 21 and, concurrently, a sequency of vestibular stimuli 22 is generated to evoke in the user 21 the vestibular stimulation felt by the athlete performing the high dive.

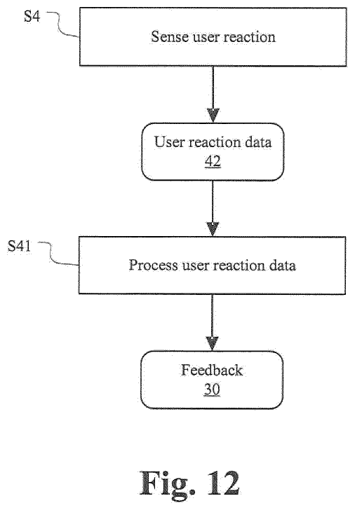

FIG. 12 illustrates an algorithm that can be used to provide feedback 30 according to one embodiment of the present disclosure. In a first step S4, which can be the same as step S4 in FIG. 5, a user reaction is sensed. A user reaction is any reaction by the user that may be connected to the application of a vestibular stimulus 22. The user reaction may, for example, be a turning of the head or body according to the vestibular stimulus 22. The user reaction may also be a movement through the surrounding. The user reaction may further be an utterance or a sound. The user reaction may also be a stumbling movement. The user reaction may further be a change in biometric measures. The sensing can be accomplished with appropriate sensing devices included into the circuitry. Sensing devices can include one or more cameras and/or EEG, and/or body trackers. Sensing devices may also include an accelerometer, which provides information on an acceleration, imaging devices or video capturing devices, which provide image information and may be used to track the user's 21 pose 25 and/or movement in the surrounding, acoustic sensors, which provide information on the surrounding or utterances of the user 21 or rotational sensors, which provide information on a rotational movement. However, the user reaction can also be sensed by biometric sensors, for example a measure of skin conductivity or a pulse of the user 21 may be included in the user reaction. Returning to FIG. 12, the sensing of the user reaction in Step S4 results in user reaction data 42, which can include data as provided by the measurements described hereinabove. In a second step S41, the user reaction data is processed to provide feedback to the user 21. This step may include computing, from the user reaction data 42, a correction factor for the strength of the vestibular stimulus 33 in a future vestibular stimulus. This step may further include computing, from the user reaction data 42, a correction to an orientation of the vestibular stimulus 22. This step may also include a determination, of the basis of the user reaction data, that generation of a vestibular stimulus 22 should cease. The latter would be advisable if, from the user reaction data, it is determined that the user 21 is in distress or that the strength of the vestibular stimulus 33 is exceedingly high.

This step can further include comparing the user reaction with an intended sports motion and the feedback 30 can include giving the user feedback on whether their reaction differs from the sports motion. The user can, for example, practice a part or all of a sports motion of which motion sequence 29 was selected so that the timed sequences of vestibular stimuli are generated by the device and the device will, in addition to generating the vestibular stimulus 22, give the user 21 feedback on whether the sports motion was practiced correctly. The feedback 30 can then, for example, include a visual, acoustic or vibrational indicator.

The user reaction data 42 can be processed with machine learning or a deep neural network in order to generate the feedback 30 in the second step S41.

It should be noted that the user reaction data 42 may not necessarily be acquired by sensors included in the circuitry but may, instead, be acquired from an outside source and then transmitted to the circuitry remotely.

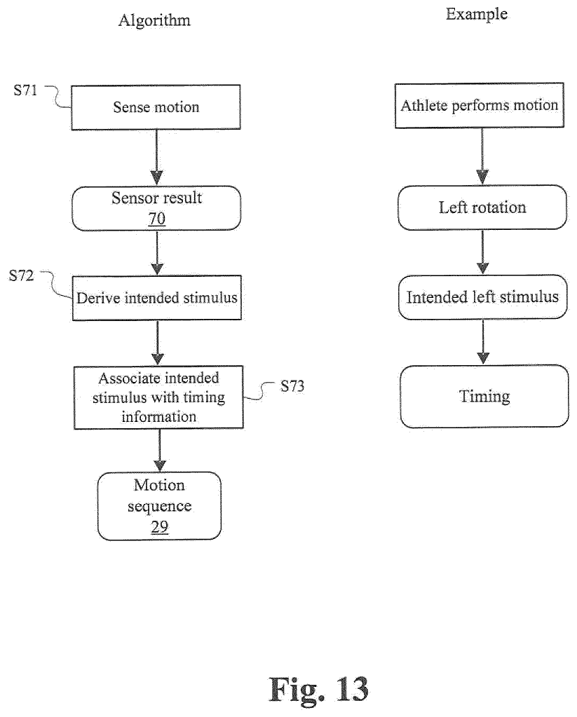

FIG. 13 illustrates a method for acquiring the timed sequences of intended stimuli 24 included in the motion sequences 29 according to one embodiment. Shown are in particular an algorithm and an example. Herein, an athlete is equipped with a sensing apparatus and data storage device. The sensing apparatus includes sensors capable of sensing a motion of the athlete. Sensing devices can include one or more cameras and/or EEG, and/or body trackers. Sensing devices may also include an accelerometer, which provides information on an acceleration, imaging devices or video capturing devices, which provide image information and may be used to track the athlete's pose 25 and/or movement in the surrounding. The sensing device may sense an acceleration and/or a rotation of the athlete's head. The athlete is then asked to perform a sports motion. As described hereinabove, the sports motion may, for example, be a short motion such as swinging a golf club, a rotation during high diving or throwing a bowling ball. The motion can also be a longer motion such as an aerobatics routine, a car race or alpine skiing or more. The motion of the athlete is then sensed in a first step S71 and stored in a sensor result 70. In a second step S72, the sensor result is analyzed and an intended stimulus 24 is derived. The derivation of the intended stimulus 24 may be a naïve algorithm, for example, of, on a abasis of the sensor result 70, the athlete is found to be rotating left, then an intended stimulus 24 of “left rotation” is derived. This step can, however, make use of an appropriate machine learning algorithm or deep neural network. The sensor the motion is associated with timing information S73. At a time T1 after the start of the sensing step, in a time window associated with T1 that may, for example, the athlete may be found to be rotating left. Then the timing T1 is associated with an intended stimulus 24 of “left rotation”. Sensing and association is repeated for successive timings until the end of the sports motion. As described hereinabove, the timing can, in addition, be associated with intended strengths of the stimuli 31. Thus, a timed sequence of intended stimuli 24 is created that can be included in the control information 27 or in the motion sequences 29. The thus created control information 27 or motion sequences 29 can then be used to generate the vestibular stimuli 22 as, for example, described in FIG. 9 or FIG. 10.

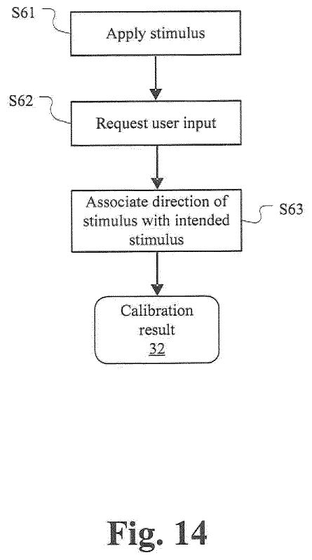

FIG. 14 illustrates an algorithm to generate a calibration 32 according to one embodiment. A calibration 32 is used in some embodiments to associate the intended stimulus 24 with the vestibular stimulus 22. In a first step S61, a vestibular stimulus 22 is generated. In a second step S62, user input is requested. For example, the user 21 may, by means of an audio signal or a message displayed on an output device, be asked “which direction do you feel you are rotating in?” and a range of options given. The user 21 may then, for example, indicate a sense of rotation in a left direction. In a third step S63 the user input and the stimulus are associated, leading to a calibration 32. In the context of the calibration 32, the acquired user input can be seen as the intended stimulus 24. In the current example a left stimulus 22L may be applied and the user may indicate a rotation in a left direction, leading to a calibration 32 that associates a left stimulus 22L with an intended stimulus 24 of left rotation. Thus, if the control information 27 or motion sequence 29 requires an intended stimulus 24, the calibration 32 indicates an appropriate vestibular stimulus 22 to be generated as shown, for example in FIG. 9 or FIG. 10.

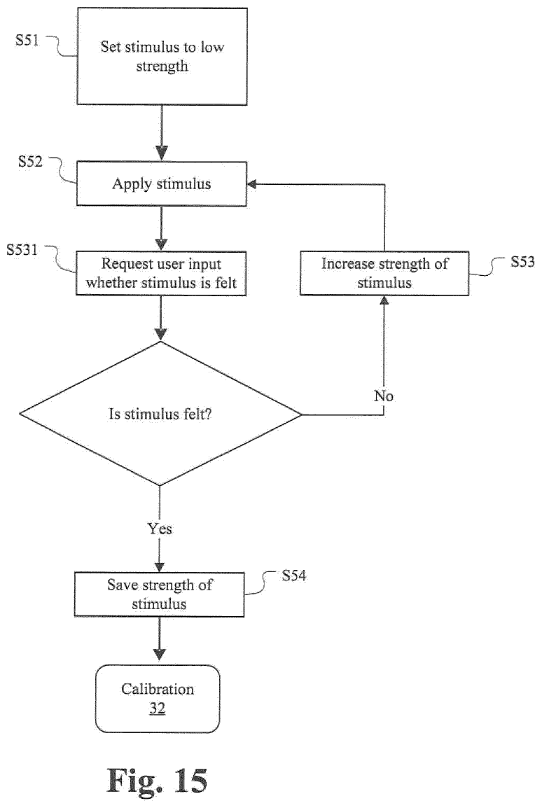

FIG. 15 illustrates another method to obtain the calibration 32 by requesting user input according to one embodiment. The following method can be executed in a separate calibration mode. Here, in a first step S51, a low value for the strength of the vestibular stimulus 33 is chosen at a low value or at zero. The vestibular stimulus 22 is generated in a second step S52. The vestibular stimulus 22 can, for example, be a vestibular stimulus 22L in a left direction. In a third step S531 the user 21 is requested to provide input to determine whether the vestibular stimulus 22 is felt. For example, the user 21 can be asked, via, for example, an audio message “do you feel like you are rotating?”. The user 21 can then, via an appropriate input device or method, enter information to indicate “yes” if the vestibular stimulus 22 is felt or “no” if the vestibular stimulus 22 is not felt. If the vestibular stimulus 22 is not felt, then, in a fourth step S53, the strength of the vestibular stimulus 33 is increased, though the maximum strength of the vestibular stimulus should not be exceeded. The vestibular stimulus 22 is then applied and user input requested again in steps S52 and S531. If the vestibular stimulus 22 is felt, indicated by “yes”, the strength of the vestibular stimulus is saved in a fifth step S54, yielding, in this embodiment, the calibration 32. It should be noted that, in this embodiment, the saved strength of the vestibular stimulus can, for example be used to compute a correction factor for the strength of the vestibular stimulus 33 to be applied. This way, an exceedingly strong vestibular stimulus can be avoided if different users 21 exhibit different levels of sensitivity to vestibular stimulation.

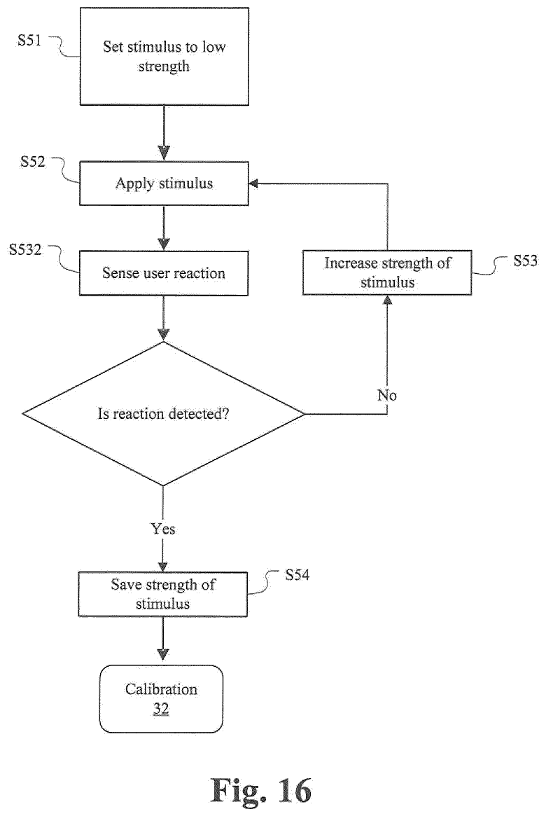

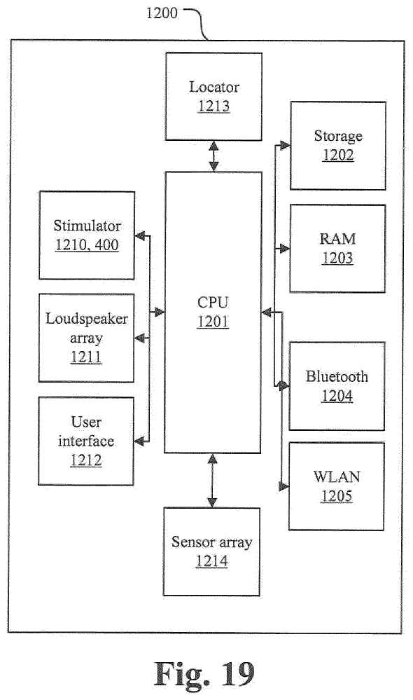

FIG. 16 illustrates a method to obtain the calibration 32 by sensing a user reaction according to one embodiment. The following method can be executed in a separate calibration mode. Here, in a first step S51, the strength of the vestibular stimulus 33 is chosen at a low value or at zero. The vestibular stimulus 22 is applied in a second step S52. The vestibular stimulus 22 can, for example, be a vestibular stimulus 22L in a left direction. In a third step S532 the user 21 can also be requested to provide input to determine whether the vestibular stimulus 22 is felt. The user reaction, which may include the same or similar measurements as in obtaining the feedback 30, as shown in FIG. 19, is measured in the third step S532. If the user reaction is not detected, indicated by “no”, then, in a fourth step S53, the strength of the vestibular stimulus 33 is increased, though the maximum strength of the vestibular stimulus should not be exceeded. The vestibular stimulus 22 is then applied and the user reaction sensed again in steps S52 and S32. If user reaction is detected, indicated by “yes”, the strength of the vestibular stimulus is saved in a fifth step S54, yielding, in this embodiment, the calibration 32. It should be noted that, in this embodiment, the saved strength of the vestibular stimulus can, for example be used to compute a correction factor for the strength of the vestibular stimulus 33 to be applied. This way, an exceedingly strong vestibular stimulus can be avoided if different users 21 exhibit different levels of sensitivity to vestibular stimulation.

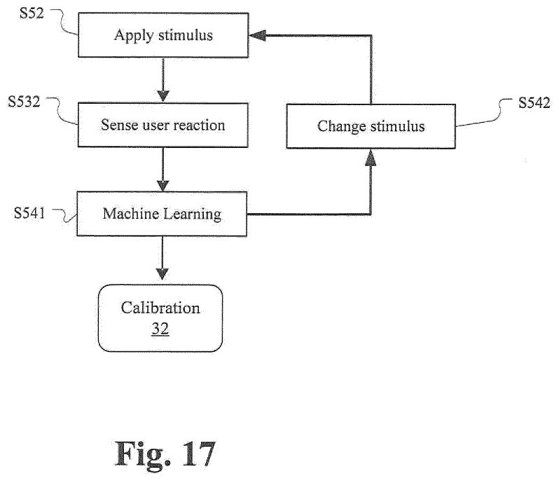

FIG. 17 illustrates a method to obtain the calibration 32 by sensing a user reaction and using a machine learning algorithm or deep neural network according to one embodiment. Here, an initial vestibular stimulus, which may or may not be at a low strength, is applied in a first step S52. In a second step S532, the user reaction, which may include the same or similar measurements as in obtaining the feedback 30 is sensed. The user reaction can then be provided to a machine learning algorithm or a deep neural network in step S541. The method can then be repeated in subsequent step S542, wherein the stimulus is changed, leading again to step S52, until the machine learning algorithm is sufficiently trained or configured to produce the calibration result 532. This method may be executed either during a dedicated calibration mode or may be used during normal operation in conjunction with step S4 in FIG. 6.

It should be noted that the methods shown in FIG. 15, FIG. 16 and FIG. 17 do not have to be applied exclusively but may be combined or used in sequence or in parallel in order to obtain the calibration 32. For example, an initial calibration according to the method shown in FIG. 15 may be executed, which is then followed by a second calibration step according to the method shown in FIG. 16, which is then augmented by the method shown in FIG. 17 during normal operation.

It is reiterated that the calibration 32 can include both associations of intended stimuli 24 and vestibular stimuli 22 and associations of intended strengths of vestibular stimuli 31 and strengths of vestibular stimuli 33.

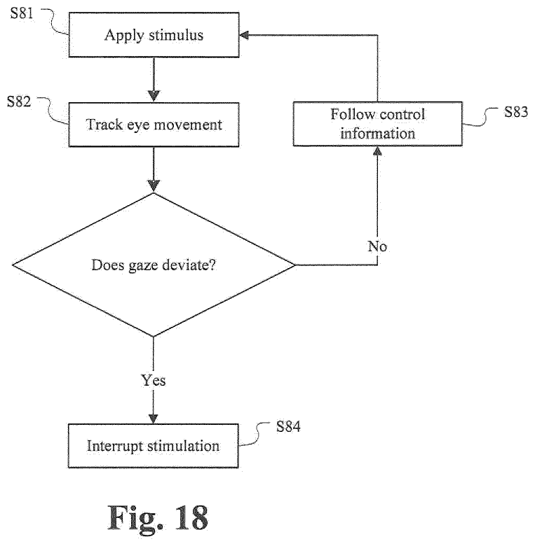

FIG. 18 shows a method to further control the generation of the stimulus 22 based on an eye movement of the user. In a first step S81, the stimulus 22 is generated. In a second step S82, the eye movement of the user 21 is tracked using, for example, eye tracking means implemented into a VR or XR device or headset. If the eye movement does not deviate from, for example, the center of a field of view of the user 21, the generation of the stimulus 22 is continued in a third step S83. If, on the other hand, the eye movement does deviate, the generation of the stimulus 22 is interrupted. This can allow the user 21 to intuitively control the stimulation. As eye movement is also correlated if feelings of discomfort, discontinuation of the stimulation based on eye movement can also prevent the user from feeling such discomfort, if caused by the stimulation, for prolonged periods.

FIG. 19 shows a general configuration of a device 1200 according to the present disclosure. The device 1200 can include a CPU 1201, interacting with storage 1202. The storage 1202 can, for example, be a solid state disk. The device 1200 can further include a read-only-memory (RAM) 1203 interacting with the CPU 1202. The device can include a Bluetooth transceiver and decoder 1204 and an antenna and circuitry configured to interface with a wireless local area network (WLAN) 1205. The circuitry contains a stimulator 400, which can also be called a stimulation unit 1210, is configured to generate the vestibular stimulus 22 according to the embodiments described hereinabove. The circuitry can further include a loudspeaker array 1211 capable of producing audible signals and a user interface 1212. The circuitry can further include a locator 1213, capable of determining a pose 25 according to the embodiments described hereinabove. The circuitry can further include a sensor array 1214 capable of sensing the user reaction and providing the user reaction data 42 according to the embodiments described hereinabove.

It should be recognized that the embodiments describe methods with an exemplary ordering of method steps. The specific ordering of method steps is however given for illustrative purposes only and should not be construed as binding. For example the ordering of S92 and S93 in the embodiment of FIG. 15 may be exchanged. Also, the ordering of S21, S23 in the embodiment of FIG. 14 may be exchanged. Other changes of the ordering of method steps may be apparent to the skilled person.

Please note that the division into units 1210 to 1214 is only made for illustration purposes and that the present disclosure is not limited to any specific division of functions in specific units. For instance, 1212 could be implemented by a respective programmed processor, field programmable gate array (FPGA) and the like.

All units and entities described in this specification and claimed in the appended claims can, if not stated otherwise, be implemented as integrated circuit logic, for example on a chip, and functionality provided by such units and entities can, if not stated otherwise, be implemented by software.

In so far as the embodiments of the disclosure described above are implemented, at least in part, using software-controlled data processing apparatus, it will be appreciated that a computer program providing such software control and a transmission, storage or other medium by which such a computer program is provided are envisaged as aspects of the present disclosure.

Note that the present technology can also be configured as described below.
(1) An electronic device comprising circuitry 1200 configured to generate a vestibular stimulus 22 based on motion sequences 29 included in control information 27 and to perform vestibular stimulation based on the vestibular stimulus 22.
(2) The electronic device according to (1), wherein the control information 27 includes timing information.
(3) The electronic device according to of any of (1) or (2), wherein the circuitry 1200 is configured to hold the control information 27; and wherein the motion sequences 29 include information on sequences of intended stimuli 24 that controls the generation of the vestibular stimulus 22.
(4) The electronic device according to of any of (1) to (3), wherein the motion sequences 29 can be selected by a user input or based on user preference information.
(5) The electronic device according to of any of (1) to (4), wherein the circuitry 1200 is further configured for the user to perform motion rehearsal and/or accustomization based on vestibular stimulation 22.
(6) The electronic device according to of any of (1) to (5), wherein the motion sequences 29 describe a difficult and/or technical motion and the motion is a sports motion.
(7) The electronic device according to of any of (1) to (6), wherein the motion sequences 29 are obtained by inertial measurement of the movement and/or the acceleration of the motion of an athlete.
(8) The electronic device according to of any of (1) to (7), wherein the vestibular stimulation is a calibrated vestibular stimulation.
(9) The electronic device according to of any of (1) to (8), wherein the circuitry 1200 is configured to further compute the calibrated vestibular stimulus based on a calibration 32 obtained from the user 21.
(10) The electronic device according to of any of (1) to (9), wherein the circuitry 1200 is further configured to obtain the calibration 32 using machine learning.
(11) The electronic device according to of any of (1) to (10), wherein the circuitry 1200, is configured to generate the vestibular stimulus 22 at various levels of intensity.
(12) The electronic device according to of any of (1) to (11), wherein the circuitry 1200 is configured to further track position and/or posture of a user with at least one camera and/or EEG, and/or body trackers to estimate the pose 25 of the user.
(13) The electronic device according to of any of (1) to (12), wherein the circuitry 1200 is configured to further control the generation of the vestibular stimulus 22 based on an eye movement.
(14) The electronic device according to of any of (1) to (13), wherein the circuitry 1200 is further configured to interrupt the stimulation based on the eye movement.
(15) The electronic device according to of any of (1) to (14), wherein the motion sequences 29 are obtained during a training process in advance.
(16) The electronic device according to of any of (1) to (15), wherein the motion sequences 29 are obtained by machine learning.
(17) The electronic device according to of any of (1) to (16), wherein the circuitry 1200 is further configured to cause a video display apparatus to display, to the user, video information associated with the motion sequences 29.
(18) The electronic device according to of any of (1) to (17), wherein the circuitry 1200 is further configured to stimulate, with the vestibular stimulus 22, at least one semicircular canal 201 of the user's 21 vestibular system 200; or at least one otolithic organ of the user's vestibular system 200; or at least one semicircular canal 201 and at least one otolithic organ of the user's 21 vestibular system 200.
(19) The electronic device according to of any of (1) to (18), wherein the circuitry 1200 is further configured to stimulate the user's 21 vestibular system 200 by at least one of a direct current stimulation, voltage stimulation, electromagnetic or magnetic stimulation, ultrasonic stimulation or direct nervous or vestibular stimulation.
(20) The electronic device according to of any of (1) to (19), wherein the circuitry 1200 comprises an EEG device or body-tracking means configured to sense the user reaction.
(21) The electronic device according to of any of (1) to (20), wherein the circuitry 1200 is provided in one or more head-mounted casings to be worn by the user.
(22) The electronic device according to of any of (1) to (21), wherein the circuitry 1200 is provided in one or more individual casings, or is incorporated into headphones, or is incorporated into a virtual-reality headset, or is incorporated into an extended-reality device.
(23) A method comprising: generate a vestibular stimulus 22 based on motion sequences 29 included in control information 27 and to perform vestibular stimulation based on the vestibular stimulus 22.

LIST OF REFERENCE SIGNS
21 User
22 Vestibular stimulus
22L Left stimulus
22R Right stimulus
24 Intended stimulus
25 Pose
27 Control information
29 Motion sequence
30 Feedback
31 Intended strength of the stimulus
32 Calibration
33 Strength of the stimulus
42 User reaction data
100 Cranium
200 Vestibular system
201 Semicircular canal
210 Vestibular hair
220 Fluid
250 Channel-like tubes
251 Wall
400 Stimulator
400L Left stimulator
400R Right stimulator
1200 Circuitry
1201 CPU
1202 Storage
1203 RAM
1204 Bluetooth
1205 WLAN
1210 Stimulation unit
1211 Loudspeaker array
1212 User interface
1213 Locator
1214 Sensor array

1. An electronic device comprising circuitry configured to generate a vestibular stimulus based on motion sequences included in control information and to perform vestibular stimulation based on the vestibular stimulus.

2. The electronic device of claim 1, wherein the control information includes timing information.

3. The electronic device of claim 1, wherein the circuitry is
configured to hold the control information; and
wherein the motion sequences include information on sequences of intended stimuli that controls the generation of the vestibular stimulus.

4. The electronic device of claim 3, wherein the motion sequences can be selected by a user input or based on user preference information.

5. The electronic device of claim 1, wherein the circuitry is further configured for the user to perform motion rehearsal and/or accustomization based on vestibular stimulation.

6. The electronic device of claim 1, wherein the motion sequences describe a difficult and/or technical motion and the motion is a sports motion.

7. The electronic device of claim 1, wherein the motion sequences are obtained by inertial measurement of the movement and/or the acceleration of the motion of an athlete.

8. The electronic device of claim 1, wherein the vestibular stimulation is a calibrated vestibular stimulation.

9. The electronic device of claim 8, wherein the circuitry is configured to further compute the calibrated vestibular stimulus based on a calibration obtained from the user.

10. The electronic device of claim 8, wherein the circuitry is further configured to obtain the calibration using machine learning.

11. The electronic device of claim 1, wherein the circuitry, is configured to generate the vestibular stimulus at various levels of intensity.

12. The electronic device of claim 1, wherein the circuitry is configured to further track position and/or posture of a user with at least one camera and/or EEG, and/or body trackers to estimate the pose of the user.

13. The electronic device of claim 1, wherein the circuitry is configured to further control the generation of the vestibular stimulus based on an eye movement.

14. The electronic device of claim 1, wherein the circuitry is further configured to interrupt the stimulation based on the eye movement.

15. The electronic device of claim 1, wherein the motion sequences are obtained during a training process in advance.

16. The electronic device of claim 1, wherein the motion sequences are obtained by machine learning.

17. The electronic device of claim 1, wherein the circuitry is further configured to cause a video display apparatus to display, to the user, video information associated with the motion sequences.

18. The electronic device of claim 1, wherein the circuitry is further configured to stimulate, with the vestibular stimulus, at least one semicircular canal of the user's vestibular system; or at least one otolithic organ of the user's vestibular system; or at least one semicircular canal and at least one otolithic organ of the user's vestibular system.

19. The electronic device of claim 1, wherein the circuitry is further configured to stimulate the user's vestibular system by at least one of a direct current stimulation, voltage stimulation, electromagnetic or magnetic stimulation, ultrasonic stimulation or direct nervous or vestibular stimulation.

20. The electronic device of claim 12, wherein the circuitry comprises an EEG device or body-tracking means configured to sense the user reaction.

21. The electronic device of claim 1, wherein the circuitry is provided in one or more head-mounted casings to be worn by the user.

22. The electronic device of claim 21, wherein the circuitry is provided in one or more individual casings, or is incorporated into headphones, or is incorporated into a virtual-reality headset, or is incorporated into an extended-reality device.

23. A method comprising:
generate a vestibular stimulus based on motion sequences included in control information and to perform vestibular stimulation based on the vestibular stimulus.
